(19) United States
(12) Patent Application Publication
(10) Pub. No.: US 2025/0134684 A1
(43) Pub. Date: May 1, 2025

(54) STENT WITH STRUTS DEFINING CIRCUMFERENTIALLY OFFSET APICES

(71) Applicant: Covidien LP, Mansfield, MA (US)

(72) Inventors: Ziang Liu, Shanghai (CN); Ramesh V. Marrey, Mission Viejo, CA (US); Ashok Nageswaran, Irvine, CA (US); Yihan Wang, Shanghai (CN); Xiaojing Guo, Shanghai (CN); Chao Zeng, Shanghai (CN); Fengyang Liu, Shanghai (CN); Edwin Y. Wang, Tustin, CA (US)

(21) Appl. No.: 18/926,460

(22) Filed: Oct. 25, 2024

(30) Foreign Application Priority Data
Oct. 26, 2023 (WO) PCT/CN2023/126710

(51) Int. Cl. A61F 2/94

(52) U.S. Cl. CPC A61F 2/94

(57) ABSTRACT

A stent includes a plurality of unit cells. Within each unit cell, a first set of struts define a first plurality of apices and a second set of struts define a second plurality of apices, wherein a first strut and a second strut of the first set of struts define a first apex with a first apex angle, and the second strut and a third strut of the first set of struts define a second apex with a second, different apex angle. Within each unit cell, a fourth strut and a fifth strut of the second set of struts define a third apex with the first apex angle, and the fifth strut and the sixth strut define a fourth apex with the second apex angle. Each apex of the first plurality of apices is circumferentially offset from another apex of the second plurality of apices.

### CROSS-REFERENCE TO RELATED APPLICATIONS

This application claims the benefit of and priority to PCT Patent Application Serial No. PCT/CN2023/126710 filed Oct. 26, 2023, the entire disclosure of which is incorporated by reference herein.

### TECHNICAL FIELD

This disclosure relates to a medical stent.

### BACKGROUND

Stents are widely used for numerous medical applications where the stent is placed in a body lumen of a patient and expanded. Stents may be used in coronary or other vasculature of the patient, as well as other body lumens. Commonly, stents are cylindrical members. Stents expand from reduced diameters to enlarger diameters. Stents may either by self-expanding or balloon-expandable. At a target location within the body lumen of the patient, the stent is expanded to substantially retain or expand the diameter of body lumen at the target location. When stents are placed in certain parts of the body, the stent may be both strong and flexible. For example, when a stent is placed within a patient's vasculature at or near a patient's joint or at a curvature within the body lumen, the stent may be bent or maintained in a bent configuration.

### SUMMARY

A stent may be formed from a plurality of rows of struts. Adjacent rows of struts may be connected by rows of connectors. When the stent is radially expanded, the struts are placed in apposition with a surface of the body lumen e.g., to retain or expand the diameter of the body lumen. Each row of struts may define a plurality of apices. Apices may facilitate collapse and expansion of the rows of struts as the stent transitions between a collapsed configuration and an expanded configuration. Apices may be defined as peaks extending towards a distal end of the stent and valleys extending towards a proximal end of the stent. When a stent is placed at a curvature within a body lumen of the patient and/or is caused to bend (e.g., in response to movement of the patient), a portion of the stent is compressed and another portion of the stent is expanded.

As the stent is compressed, peaks and valleys of adjacent rows of strut may be brought into contact with each other. For example, the peaks of a first row of struts may contact the valleys of a second row of struts when the stent is compressed. The contact between the peaks and valleys may lead to protrusion of struts into the body lumen and/or interference between the peaks and valleys. The protrusions and interference may decrease an amount of apposition between the surface of the body lumen, which may affect fluid flow through the body lumen, e.g., as a result of development of thrombosis within the body lumen.

The disclosure describes example devices, systems, and methods for increasing apposition of struts of a stent to a surface of a body lumen. Example stents described herein may include rows of struts with circumferentially offset peaks and valleys. When the stent is compressed, the circumferential offset between the peaks and valleys reduce interference between the struts of the stent in response to the compression, e.g., by causing the struts to extend past, rather than into, one another. In some examples, the struts of adjacent rows and connectors of the stent define unit cells. Within each unit cell, the struts may define apices with different apex angles. The different apex angles cause the peaks and valleys within each unit cell to be circumferentially offset, e.g., without altering the boundaries of the unit cell. In some examples, the rows of struts may define a helical pattern along the longitudinal length of the stent, which may cause the peaks and valleys of adjacent rows of struts to become circumferentially offset.

The example devices, systems, and methods described in this disclosure may provide several advantages over other medical stents. Stents with the example strut patterns described herein may increase apposition of the stent to the body lumen of the patient without compromising the flexibility and/or the strength of the stent. The stents described herein may also increase apposition of the stent to the body lumen without causing the stent to experience increased torque during expansion of the stent, thereby simplifying the stent deployment process.

In some examples, this disclosure describes a stent comprising: a plurality of rows of struts extending along the longitudinal axis, each row of struts of the plurality of rows of struts extending around the longitudinal axis; and a plurality of rows of connectors, each row of connectors of the plurality of connectors extending between longitudinally adjacent rows of struts; wherein the plurality of rows of struts and the plurality of rows of connectors define a plurality of unit cells, wherein each unit cell is defined by: a first set of struts of a first row of struts of the plurality of rows of struts, a second set of struts of a second row of struts of the plurality of rows of struts, and two circumferentially adjacent connectors of a row of connectors of the plurality of rows, the row of connectors being between the first row of struts and the second row of struts along the longitudinal axis, wherein within each unit cell, the first set of struts define a first plurality of apices and the second set of struts define a second plurality of apices, wherein within each unit cell, a first strut and a second strut of the first set of struts define a first apex with a first apex angle, and the second strut and a third strut of the first set of struts define a second apex with a second apex angle, the second apex angle being less than the first apex angle, and wherein within each unit cell, a fourth strut and a fifth strut of the second set of struts define a third apex with the first apex angle, and the fifth strut and the sixth strut define a fourth apex with the second apex angle.

In some examples, this disclosure describes a method comprising: receiving, by a subtractive manufacturing assembly, fabrication data for manufacturing a stent, the stent comprising: a plurality of rows of struts extending along the longitudinal axis, each row of struts of the plurality of rows of struts extending around the longitudinal axis; and a plurality of rows of connectors, each row of connectors of the plurality of connectors extending between longitudinally adjacent rows of struts; wherein the plurality of rows of struts and the plurality of rows of connectors define a plurality of unit cells, wherein each unit cell is defined by: a first set of struts of a first row of struts of the plurality of rows of struts, a second set of struts of a second row of struts of the plurality of rows of struts, and two circumferentially adjacent connectors of a row of connectors of the plurality of rows, the row of connectors being between the first row of struts and the second row of struts along the longitudinal axis, wherein within each unit cell, the first set of struts define a first plurality of apices and the second set of struts define a second plurality of apices, wherein within each unit cell, a first strut and a second strut of the first set of struts define a first apex with a first apex angle, and the second strut and a third strut of the first set of struts define a second apex with a second apex angle, the second apex angle being less than the first apex angle, and wherein within each unit cell, a fourth strut and a fifth strut of the second set of struts define a third apex with the first apex angle, and the fifth strut and the sixth strut define a fourth apex with the second apex angle; and removing, by the subtractive manufacturing assembly, material from an elongated body to form the stent.

In some examples, this disclosure describes a method comprising: advancing a stent within a body lumen of a patient to a target location, wherein the stent comprises: a plurality of rows of struts extending along the longitudinal axis, each row of struts of the plurality of rows of struts extending around the longitudinal axis; and a plurality of rows of connectors, each row of connectors of the plurality of connectors extending between longitudinally adjacent rows of struts; wherein the plurality of rows of struts and the plurality of rows of connectors define a plurality of unit cells, wherein each unit cell is defined by: a first set of struts of a first row of struts of the plurality of rows of struts, a second set of struts of a second row of struts of the plurality of rows of struts, and two circumferentially adjacent connectors of a row of connectors of the plurality of rows, the row of connectors being between the first row of struts and the second row of struts along the longitudinal axis, wherein within each unit cell, the first set of struts define a first plurality of apices and the second set of struts define a second plurality of apices, wherein within each unit cell, a first strut and a second strut of the first set of struts define a first apex with a first apex angle, and the second strut and a third strut of the first set of struts define a second apex with a second apex angle, the second apex angle being less than the first apex angle, and wherein within each unit cell, a fourth strut and a fifth strut of the second set of struts define a third apex with the first apex angle, and the fifth strut and the sixth strut define a fourth apex with the second apex angle; and expanding the stent radially outwards of the longitudinal axis at the target location to place the struts of the plurality of rows of struts in apposition with a surface of the body lumen at the target location.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

FIG. 1 is a conceptual diagram illustrating an example stent in an expanded configuration within a body lumen of a patient.

FIG. 2 is a conceptual diagram illustrating an example of a unit cell of the example stent of FIG. 1 in a collapsed configuration.

FIG. 3 is a conceptual diagram illustrating the example unit cell of FIG. 2 in an expanded configuration.

FIG. 4 is a cross-section diagram illustrating a cross-sectional view of an example row of struts of the stent of FIG. 1, the cross-section being taken along line A-A of FIG. 1.

FIG. 5 is a conceptual diagram illustrating the example unit cell of FIG. 2 in the expanded configuration when the stent is compressed.

FIG. 6 is a conceptual diagram illustrating another example stent in an expanded configuration.

FIG. 7 is a conceptual diagram illustrating example rows of struts of the example stent of FIG. 6.

FIG. 8A is a conceptual diagram illustrating an example row of struts of FIG. 6, the example row has been longitudinally cut and laid flat.

FIG. 8B is a conceptual diagram illustrating another view of the example row of struts of FIG. 8A.

FIG. 9A is a conceptual diagram illustrating an example unit cell of FIG. 6 in the collapsed configuration.

FIG. 9B is a cross-sectional diagram illustrating a cross-section view of the example unit cell of FIG. 9B, the cross-section being taken along line B-B of FIG. 9A.

FIG. 10 is a conceptual diagram illustrating an example end row of struts of the example stent of FIG. 6, the example row has been longitudinally cut and laid flat.

FIG. 11 is a conceptual diagram illustrating an example pair of struts of the example row of struts of FIG. 8A.

FIG. 12 is a flow chart illustrating an example process of manufacturing an example stent.

FIG. 13 is a flow chart illustrating an example process of deploying an example stent within a patient.

### DETAILED DESCRIPTION

A medical stent may by inserted within a body of a patient and deployed within a body lumen of the patient. When deployed, the stent may expand radially outwards away from a longitudinal axis of the stent and come into apposition with (i.e., contact with or proximal support of) a surface of the body lumen. When expanded, the stent may retain or expand a diameter of the body lumen.

The stent may be formed via a plurality of interconnecting struts. The plurality of struts may define rows of struts positioned along the longitudinal length of the stent. Each row of struts may extend around the longitudinal axis of the stent and may define an outer diameter of the stent. When the stent is expanded, the struts may be placed in apposition with the surface of the body lumen, e.g., to retain or expand the diameter of the body lumen. Each row of struts may define apices connecting struts. The apices may include peaks extending towards a distal end of the stent and valleys extending towards a proximal end of the stent. The apices may facilitate transition of the stent between a collapsed configuration and an expanded configuration. Longitudinally adjacent rows of struts may be connected via connectors. Each connector may connect an apex of one row struts to another apex of a longitudinally adjacent row of struts.

Stents may be self-expandable or may be balloon-expandable, e.g., expandable via expansion of a balloon of a stent delivery system. Stents may include open-cell stents, where at least some of the apices of the stent are not connected to other apices of the stent, and closed-cell stents, wherein each apex of the stent is connected to another apex of the stent, e.g., via connectors. Compared to closed-cell stents, open-cell stents are more flexible and are easier to adhere to the surface of the body lumen, e.g., at or around a curvature in the body lumen.

When the stent is expanded at or around a curvature in the body lumen, the stent may bend or otherwise deform to contact the surface of the body lumen at or around the curvature. In such examples, a portion of the stent may compress and another portion of the stent may elongate, thereby causing the stent to assume a bent configuration, e.g., in conformity with the curvature in the body lumen. In some examples, such as with open-celled stents, apices of adjacent rows of struts may interfere with each other when at least a portion of the stent is compressed. For example, apices of adjacent rows of struts may longitudinally overlap and/or may be placed in contact with each other. In some examples, one set of struts and accompanying apices are forced away from the surface of the body lumen and protrude into the body lumen. In some examples, sets of struts and accompanying apices from adjacent rows of struts longitudinally overlap each other, thereby forming overlapping layers of struts within the body lumen and reduce apposition of the stent to the surface of the body lumen.

Decrease in apposition between the stent and the surface of the body lumen may lead to one or more medical conditions and/or may decrease efficacy of the medical treatment provided by the stent/Medical conditions may include, but are not limited to, stenosis, thrombosis, or the like. Such medical conditions may lead to clotting within the body lumen (where the body lumen is a blood vessel) or other impediments, which may reduce efficacy of the medical treatment provided by the stent.

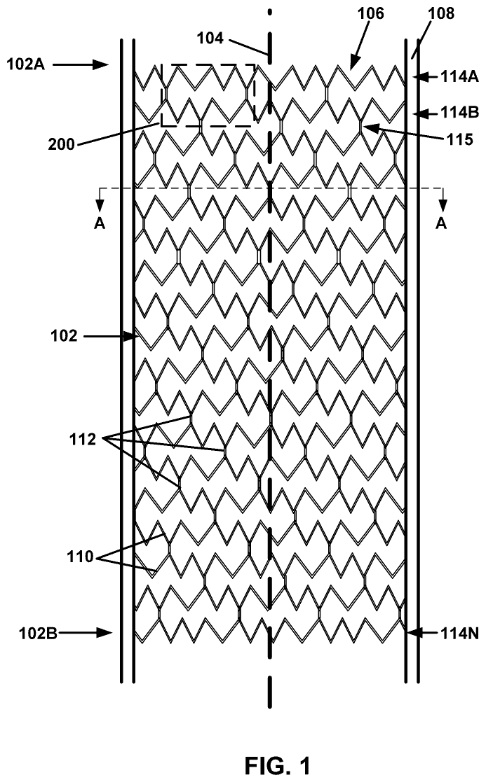

FIG. 1 is a conceptual diagram illustrating an example stent 102 in an expanded configuration within a blood vessel 106 of a patient. Stent 102 may extend from a distal end 102A to a proximal end 102B along a longitudinal axis 104 of stent 102. Stent 102 may be formed from a plurality of rows 114A-N (alternatively referred to herein as “rows 114”) of struts 110. Longitudinally adjacent rows 114 of struts 110 (e.g., along longitudinal axis 102) may be connected by a plurality of conductors 112. While FIG. 1 is described primarily with reference to blood vessel 106, stent 102 may be disposed within any other body lumen of the patient.

Stent 102 may transition between a collapsed configuration and an expanded configuration, as illustrated in FIG. 1. Stent 102 may define a larger outer diameter in the expanded configuration compared to the collapsed configuration. A clinician may navigate stent 102 to a target location within blood vessel 106 in the collapsed configuration and expand stent 102 into the expanded configuration to contact vessel wall 108 of blood vessel 106 at the target location. Stent 102 may be self-expanding or may be expanded within blood vessel 106 via an expanding element (e.g., a balloon) of a delivery element used to deliver stent 102 to the target location.

Struts 110 of stent 102 may define an outer perimeter of stent 102. When stent 102 is expanded within blood vessel 106, struts 110 may be placed in apposition with a vessel wall 108. When stent 102 is in the expanded configuration, struts 110 contact vessel wall 108 to maintain or increase a diameter of blood vessel 106 without puncturing vessel wall 108. Struts 110 may define a plurality of rows 114, each row 114 extending around longitudinal axis 104 and defining the outer perimeter of stent 102. Each of struts 110 is connected to two other struts 110 within the same row 114 at a pair of respective apices.

Each apex connects the ends of two separate struts 110 and may facilitate collapse or expansion of rows 114 of struts 110 during transition of stent 102 between the collapsed configuration and the expanded configuration. Each apex may function as a hinge between two struts 110 to facilitate collapse of struts 110 towards each other and/or expansion of struts 110 away from each other. Apices may define same or different dimensions (e.g., cross-sectional dimensions) as struts 110. For example, each apex may define a cross-sectional width greater than the cross-sectional width of a strut 110 extending from the apex. Apices may include peaks extending towards distally along longitudinal axis 104 and valleys extending proximally along longitudinal axis 104. Within each row 114, struts 110 may define alternating peaks and valleys around the circumference of the row 114. While each apex is described as “connecting” struts, rows 114 of struts 110 may be monolithic, such that the apex may be a functional interface of two adjacent struts 110.

Longitudinally alternating rows 114 of struts 110 may define alternating patterns of apices. For example, relative to a reference point around the circumference of the stent 102, a first row 114 (e.g., row 114A) may define a first alternating pattern of peaks and valleys (e.g., a peak-valley-peak pattern) and a longitudinally adjacent row 114 (e.g., row 114B) may define a different alternating pattern of peaks and valleys (e.g., a valley-peak-valley pattern). Longitudinally adjacent rows 114 of struts 110 may define different numbers of peaks and valleys. For example, first row 114A may define a first number of peaks and a second number of valleys. Second row 114B may define the second number of peaks and the first number of valleys. The first number may be greater than or less than the second number. For example, the first number may be one greater or one less than the second number.

Each row 114 of struts 110 extends around longitudinal axis 104 and forms a complete ring. In some examples, as illustrated in FIG. 1, each row 114 defines a reference plane orthogonal to longitudinal axis 104. Each row 114 may expand radially away from and/or collapse radially towards longitudinal axis 104 along the reference plane. Within each row 114, as struts 110 expand away each other (e.g., with the apices connecting struts 110 functioning as hinges) along the perimeter of the row 114, the expansions of all of struts 110 within the row 114 may cause the row 114 to expand outwards away from longitudinal axis 104. Similarly, collapse of all struts 110 along perimeter of row 114 may cause the row 114 to collapse inwards and towards longitudinal axis 104.

Rows 114 of struts 110 are connected via connectors 112. For example, one or more connectors 112 may connect struts 110 within first row 114A to struts within second row 114B. Connectors 112 may be connected to struts 110 at the apices defined by struts 110. For example, connectors 112 may connect to peaks and valleys defined by struts 110 within each row 114. One or more connectors 112 may connect valleys of one row 114 (e.g., first row 114A) to peaks of another row 114 (e.g., second row 114B), may connect valleys of different rows 114 (e.g., valleys of first row 114A and second row 114B), may connect peaks of different rows 114 (e.g., peaks of first row 114A and second row 114B), and/or may connect peaks of one row 114 (e.g., first row 114A) to valleys of another row 114 (e.g., second row 114B). Each connector 112 may be coupled to two apices defined by struts 110 within adjacent rows 114. One or more apices within stent 102 may not be coupled to another apex within stent 102 via connector 112 (e.g., one or more apices are unconnected). In such examples, stent 102 may be referred to as an open-celled stent, with unconnected apices within stent 102. Each connector 114 may extend parallel to longitudinal axis 104 or may extend along a reference axis offset from longitudinal axis 104.

Connectors 112 may define rows 115 of connectors 112, each row 115 extending around longitudinal axis 104 and forming a complete ring. Rows 115 of connectors 112 may be disposed between longitudinally adjacent rows 114 of struts 110 (e.g., between first row 114A and second row 114B). Connectors 112 within each row 115 may connect the longitudinally adjacent rows 114 of struts 110 disposed around the row 115. Connectors 112 within each row 115 may be evenly distributed around the perimeter of stent 102, e.g., to facilitate uniform and repeatable transition of stent 102 between the collapsed configuration and the expanded configuration.

Struts 110 and connectors 112 may define a plurality of unit cells 200 within stent 102. Each unit cell 200 may be defined by two circumferentially adjacent connectors 112 and struts 110 within longitudinally adjacent rows 114 and extending between the two circumferentially adjacent connectors 112. For example, as illustrated in FIG. 1, a unit cell 200 may be defined by two circumferentially adjacent connectors 112, each connector 112 connecting first row 114A to second row 114B, as well as struts 110 of first row 114A and second row 114B extending between the two connectors 112. Each row 114 may include one, two, three, four, or more unit cells 200 around the outer perimeter of stent 102.

Within each unit cell 200, struts 110 may define apices with different apex angles, e.g., apices with a first apex angle and apices with a second apex angle different from (e.g., smaller) than the first apex angle. Within each unit cell 200, at least one peak and at least one circumferentially adjacent valley in the same row 114 may have the first apex angle. In such examples, each unit cell 200 may include at least four apices with the first apex angle (e.g., with at least two apices with the first apex angle per row 114 and two rows 114 defining unit cell 200). In other examples, each unit cell 200 may include a different number of apices with the first apex angle, e.g., three or less or five or more apices with the first apex angle per unit cell 200. The different apex angles may cause apices of first row 114A and apices of second row 114B to be circumferentially offset within unit cell 200, thereby reducing or inhibiting interference between apices of first row 114A and apices of second row 114B in response to compression of unit cell 200 due to compression of stent 102, e g., in response to bending of stent 102. Different unit cells 200 along stent 102 may define uniform or different dimensions and/or characteristics (e.g., number of apices within unit cell 200, number of struts 110 within unit cell 200, apex angle value(s) within unit cell 200, length of unit cell 200 along a reference axis orthogonal to longitudinal axis 104, or width of unit cell 200 along longitudinal axis 104). In some examples, the dimensions and/or characteristics of unit cells 200 vary along longitudinal axis 104 such that unit cells 200 at or around distal end 102A of stent 102 exhibit different dimensions and/or characteristics compared to unit cells 200 at or around proximal end 102B of stent 102.

Stent 102 as illustrated in FIG. 1 may provide several advantages over other stents (e.g., other open-celled stents). Stent 102 includes struts 110 defining apices with different apex angles, which may cause the apices of adjacent rows 114 of struts 112 to be circumferential offset. The circumferential offset of apices may reduce and/or inhibit interference between the apices in response to compression of stent 102, which may provide stent 102 with increased apposition to vessel wall 108 compared to an otherwise identical stent with circumferentially aligned apices. Stent 102 as described herein may also define apices with different apex angles within unit cells 200 without altering the dimensions and/or shape of unit cells 200 as compared to unit cells within an otherwise identical stent with uniform apex angles within each unit cell. Unit cells 200 described herein may increase apposition of struts 110 to vessel wall 108 compared to the otherwise identical stent and may facilitate even distribution of unit cells 200 around the perimeter of stent 102 and/or along the longitudinal length of stent 102, thereby allowing the entirety of stent 102 to exhibit the flexibility, strength, and increased apposition properties without biasing specific regions on stent 102 and/or without imparting additional torque on stent 102 during expansion of stent 102.

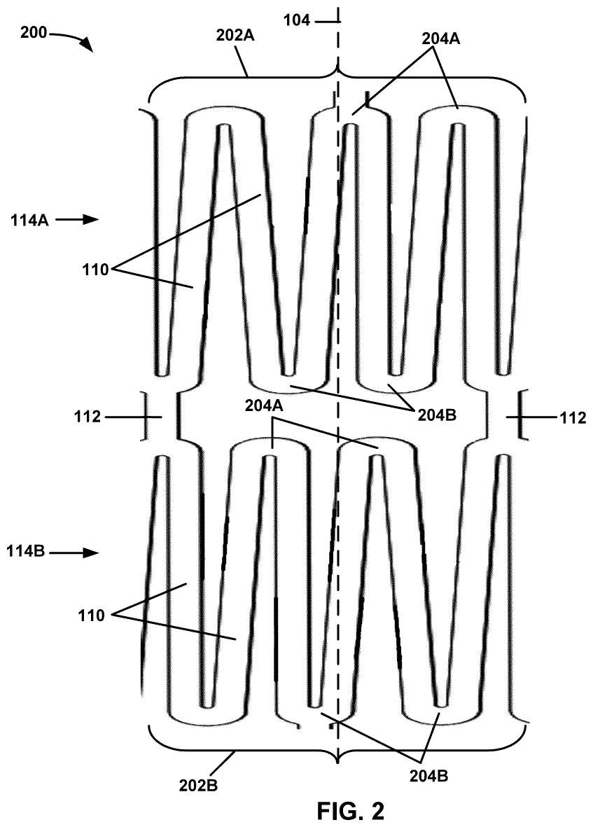

FIG. 2 is a conceptual diagram illustrating an example of a unit cell 200 of the example stent 102 of FIG. 1 in a collapsed configuration. As illustrated in FIG. 2, unit cell 200 may be defined by two circumferentially adjacent connectors 112 and sets of struts 110 from rows 114 (e.g., from rows 114A, 114B) connected by the two connectors 112. Within unit cell 200, each connector 112 couples one set of struts 110 from one row 114 (e.g., set 202A from first row 114A) to another set of struts 110 from another row 114 (e.g., set 202B from second row 114B. Connectors 112 may connect to sets 202A, 202B (collectively referred to herein as “sets 202”) at apices 204A, 204B (collectively referred to as “apices 204”).

Apices 204 may be defined relative to a reference (e.g., a distal end) to include peaks 204A and valleys 204B. Each of sets 202 of struts 110 may define a plurality of apices 204, each apex 204 connecting two circumferentially adjacent struts 110. Each of sets 202 may define apices 204 including alternating peaks 204A and valleys 204B. For example, as illustrated in FIG. 2, first set 202A may define apices 204 with a peak-valley-peak pattern within unit cell 200 while second set 202B defines apices 204 with a valley-peak-valley pattern within unit cell 200. Apices 204 may function as hinges between circumferentially adjacent struts 110 and facilitate expansion of a row of struts 110 away from the collapsed configuration and/or collapse of a row of struts 110 from the expanded configuration (e.g., as illustrated in FIG. 1) into the collapsed configuration. Within unit cell 200, apices 204 of first set 202A may be longitudinally offset from apices 204 of second set 202B.

Valleys 204B of first set 202A may be longitudinally adjacent to peaks 204A of second set 202B. The outermost valleys 204B of first set 202A within unit cell 200 may be connected to the outermost peaks 204A of second set 202B within unit cell 200 by connectors 112, e.g., thereby forming peak-to-valley connections. The peak-to-valley connections may define the outer boundaries of unit cell 200 along the outer perimeter of stent 102. Each of struts 110 and connectors 112 may define two or more unit cells 200. For example, one or more struts 110 may define two separate unit cells 200 (e.g., two adjacent unit cells 200).

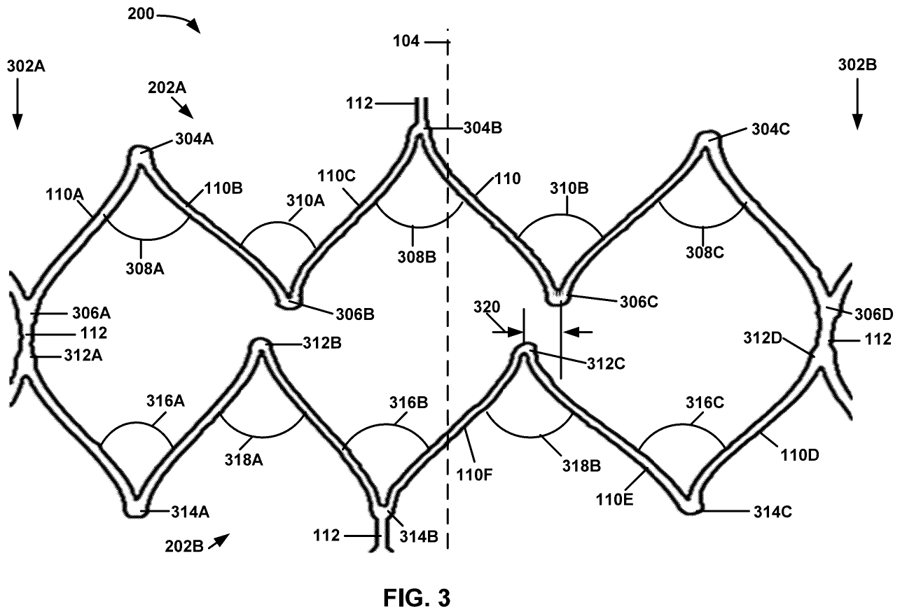

FIG. 3 is a conceptual diagram illustrating the example unit cell 200 of FIG. 2 in an expanded configuration. When expanded unit cell 200 extends from a first end 302A to a second end 302B. Connectors 112 are disposed at first end 302A and second end 302B (collectively referred to herein as “ends 302”). Within unit cell 200, first set 202A of struts 110 may define peaks 304A-C (collectively referred to herein as “peaks 304”) and valleys 306A-D (collectively referred to herein as “valleys 306”). For example, as illustrated in FIG. 3, a first strut 110A and a second strut 110B of first set 202A may define peak 304A. Second strut 110B may also define valley 306B in conjunction with a third strut 110C of first set 202A. Each of peaks 304 may define a respective apex angle of apex angles 308A-C (collective referred to herein as “apex angles 308”) and each of valleys 306 within unit cell 200 (e.g., valleys 306B, 306C) may define a respective apex angle of apex angles 310A-310B (collectively referred to herein as “apex angles 310”). Each apex angle 310 may be defined by an angle created by an axis of each of two adjacent struts 110, such as defined at a mid-point of each strut 110. Within unit cell 200, second set 202B of struts 110 may define peaks 312A-D (collectively referred to herein as “peaks 312”) and valleys 316A-C (collectively referred to herein as “valleys 316”). For example, a fourth strut 110D and a fifth strut 110E of second set 202B may define valley 314C. The fifth strut 110E and a sixth strut 110F of second set 202B may define peak 312C. Each of peaks 312 within unit cell 200 (e.g., peaks 312B, 312C) may define a respective apex angle of apex angles 318A-B (collective referred to herein as “apex angles 318”) and each of valleys 314 within unit cell 200 may define a respective apex angle of apex angles 316A-316C (collectively referred to herein as “apex angles 316”). Each of apex angles 308, 310, 316 and 318 may be up to about 90 degrees. First set 202A and second set 202B may be collectively referred to herein as “sets 202”.

Each of sets 202 may including alternating peaks and valleys, e.g., alternating peaks 304 and valleys 306 or alternating peaks 312 and valleys 314. Within unit cell 200, each of sets 202 may include a different number of peaks and valleys. For example, as illustrated in FIG. 3, unit cell 200 may include three peaks 304 (e.g., peaks 304A-C) and two valleys 306 (e.g., valleys 306B, 306C) in set 202A and two peaks 312 (e.g., peaks 312B, 312C) and three valleys 314 (e.g., valleys 314A-C) in second set 202B. In some examples, unit cell 200 may include up to three peaks and/or valleys in each set 202 of struts 110 in unit cell 200. In some example unit cell 200 may include more or fewer peaks and/or valleys in each set 202 of struts 110 in unit cell 200 (e.g., less than three or four or more peaks and/or valleys).

Connectors 112 may connect the outermost valleys 306 of first set 202A of struts 110 (e.g., valleys 306A, 306D) to the outermost peaks 312 of second set 202B of struts 110 (e.g., peaks 312A, 312D) to define ends 302A, 302B of unit cell 200. Peaks 304, 312 and valleys 306, 314 between ends 302A, 302B may define apex angles 308, 310, 316, and 318 with two or more different values to cause valleys 306 (e.g., valleys 306B, 306C) and peaks 312 (e.g., peaks 312B, 312C) within unit cell 200 to be circumferentially offset by at least distance 320. Distance 320 may be from about 0.08 to about 0.13 millimeters (mm) (e.g., about 0.003 to about 0.005 inches (in)). Distance 320 may be measured between the centers of valleys 306 and peaks 312. In other examples, distance 320 may be of different values depending at least in part on one or more factors. The one or more factors may include, but are not limited to, an outer diameter of stent 102, a number of valleys 306 and peaks 312 per unit cell 200, width(s) of struts 110, or a width of unit cell 200. Circumferential offset between valleys 306 and peaks 312 within unit cell 200 by distance 320 may reduce or inhibit interference between valleys 306 and peaks 312 in response to compression of unit cell 200 along longitudinal axis 104.

Apices 304, 306, 312, and 314 may define one of two or more different values. For example, apex angles 308A, 318A, 316C, and 318B may define a first apex angle value and apex angles 308B, 308C, 310B, 316A, 316B, 318A, may define a second apex angle value different from the first apex angle value. The first apex angle value may be greater than the second apex angle value (e.g., may be up to about 20% greater than the second apex angle value). Struts 110 defining apex angles 308, 310, 316, and 318 with the first apex angle value may define a different length than struts 110 defining apex angles 308, 310, 316, and 318 with the second apex angle.

Each of sets 202 may define apices having the first apex angle at opposing ends 302, e.g., to maintain circumferential offset between valleys 306 and peaks 312 within unit cell 200. For example, peak 304A and valley 306B with apex angles 308A, 310A and peak 312C and valley 314C with apex angles 318B, 316C may also define the first angle value. Both sets 202 may include apex angles defining the first angle value to form circumferential offset between valleys 306 and peaks within unit cell 200 while maintaining the width of unit cell 200 between ends 302, e.g., compared to another unit cell 200 with identical dimensions and including apices defining a uniform apex angle value.

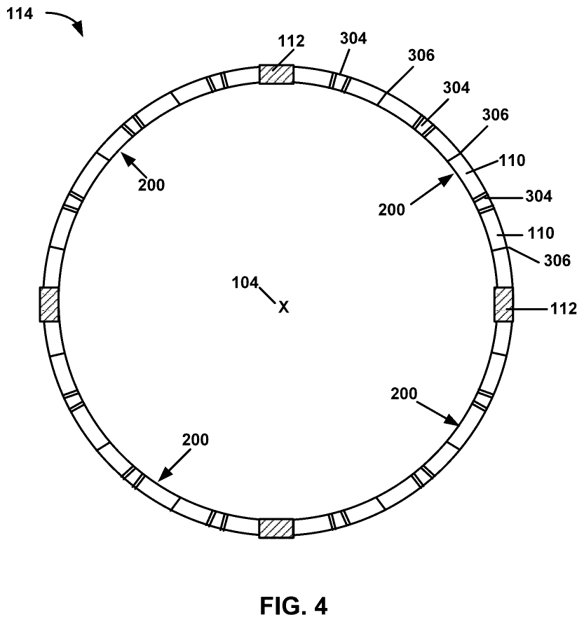

FIG. 4 is a cross-section diagram illustrating a cross-sectional view of an example row 114A of struts 110 of the stent 102 of FIG. 1, the cross-section being taken along line A-A of FIG. 1. The example row 114 of struts 110 may be defined by multiple unit cells 200 (e.g., by four or more unit cells 200). Row 114 may include a plurality of struts 110, circumferentially adjacent struts 110 being connected by apices. Apices may include peaks 304 and valleys 306. Row 114 may include struts 110 connected by alternating peaks 304 and valleys 306. Row 114 may be centrosymmetric about longitudinal axis 104, e.g., row 114 is symmetric about a central point of row 114 disposed along longitudinal axis 104. In some examples, as illustrated in FIG. 4, connectors 112 are centrosymmetric about longitudinal axis 104.

Row 114 may be connected to adjacent rows 114 via connectors 112. Connectors 112 may be equally distributed around the perimeter of row 114, e.g., to facilitate equal expansion of row 114 around the perimeter of row 114. Connectors 112 may define ends of unit cells 200. As illustrated in FIG. 4, each connector 112 may define ends of two unit cells 200, e.g., two circumferentially adjacent unit cells 200. Connectors 112 may define a circular, oval, rectangular, or other polygonal cross-section (e.g., a trapezoidal, quadrilateral, pentagonal, or hexagonal cross-section).

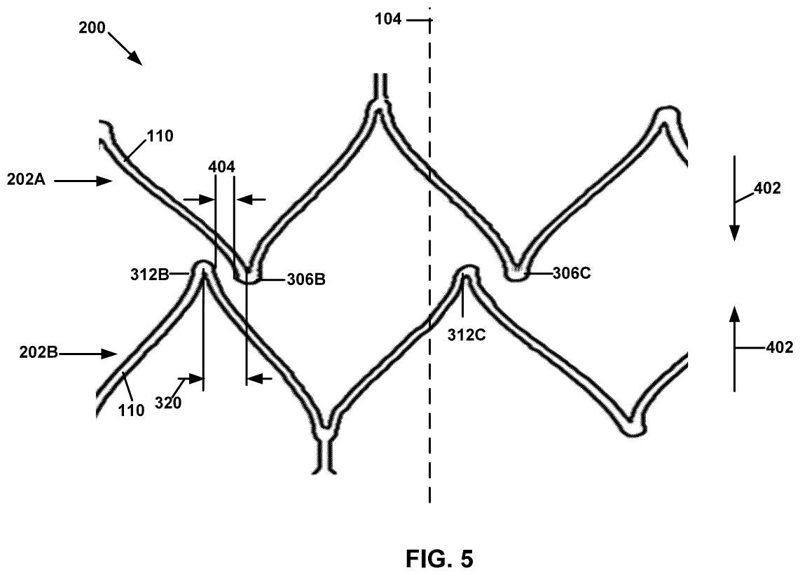

FIG. 5 is a conceptual diagram illustrating unit cell 200 of FIG. 2 in the expanded configuration when stent 102 is compressed. When stent 102 bends (e.g., within a curvature in blood vessel 106), a compressive force 402 is applied on at least some unit cells 200 of stent 102, causing the at least some unit cells 200 to compress and, by extension, portions of stent 102 to compress. Compressive force 402 may be applied on unit cell 200 along a direction parallel to longitudinal axis 104.

When unit cell 200 is compressed, valleys 306B, 306C of first set 202A of struts 110 may longitudinally overlap with peaks 312B, 312C of second set 202B of struts 110, respectively. As illustrated in FIG. 5, valleys 306B, 306C may be circumferentially offset from peaks 312B, 312C by distance 320. As a result, when valleys 306B, 306C longitudinally overlap with peaks 312B, 312C, struts 110 defining valleys 306B, 306C may be separated from struts 110 defining peaks 312B, 312C by at least distance 404. Distance 404 may up to about 0.05 mm (e.g., up to about 0.002 in).

When struts 110 defining valleys 306B, 306C are separated from struts 110 defining peaks 312B, 312C by distance 404, valleys 306B, 306C may be inhibited from interfering with peaks 312B, 312C, thereby reducing or inhibiting a potential loss of apposition of struts 110 with vessel wall 108 resulting from interference between struts 110 of first set 202A and struts 110 of second set 202B.

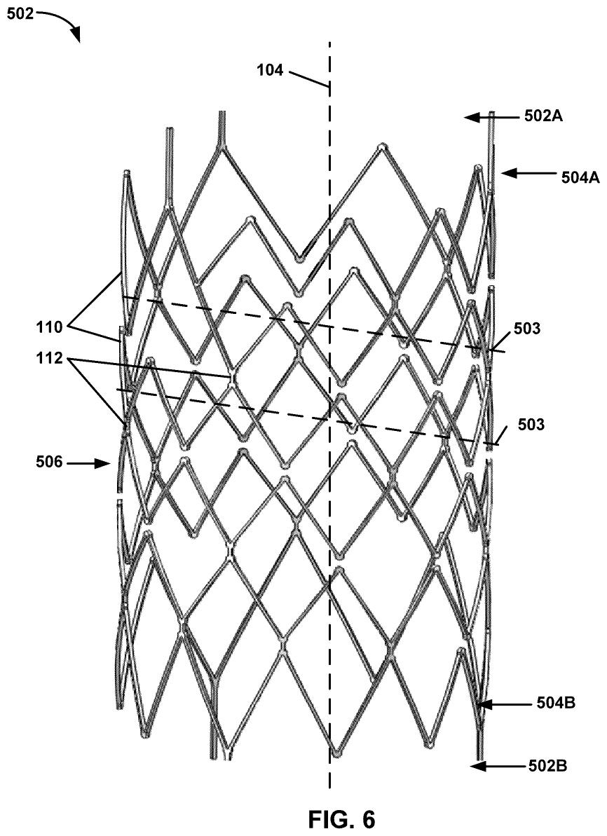

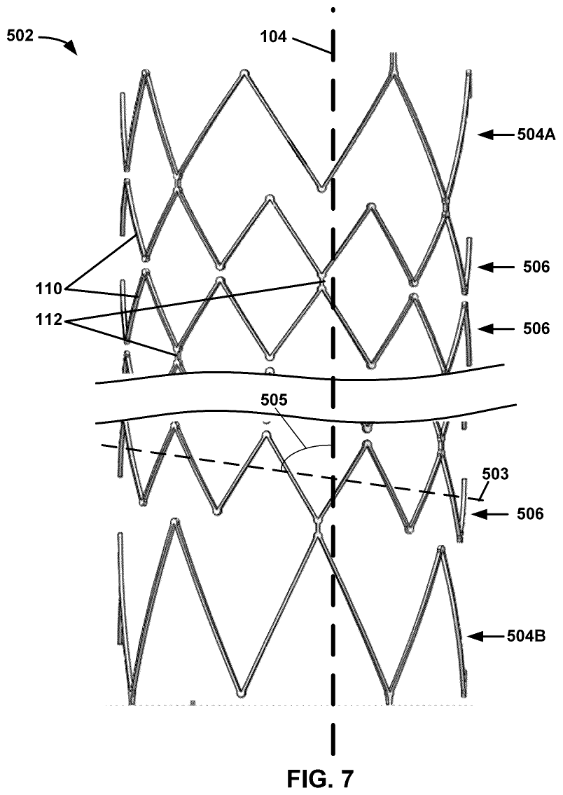

In some examples, stents described herein may include rows of stents that form a pattern, such as a helical or spiral pattern. FIG. 6 is a conceptual diagram illustrating another example stent 502 in an expanded configuration. FIG. 7 is a conceptual diagram illustrating example rows of struts of the example stent of FIG. 6. Stent 502 may extend from a distal end 502A to a proximal end 502B along longitudinal axis 104. Stent 502 may include a distal row 504A of struts 110 at or around distal end 502A of stent 502. Stent 502 may include a proximal row 504B of struts 110 at or around proximal end 502B of stent 502. Stent 502 may include a plurality of rows 506 of struts 110 disposed along longitudinal axis 104 between distal row 504A and proximal row 504B. Rows 504A, 504B, and 506 may be connected via a plurality of connectors 112 disposed connecting struts 110 of longitudinally adjacent rows. Rows 506 may define a helical or spiral pattern around the perimeter of stent 502 and along longitudinal axis 104. The helical or spiral pattern may form a circumferential offset between longitudinally adjacent apices defined by struts 110 and may inhibit or reduce interference between struts 110 when stent 502 is at least partially compressed.

One end of first row 504A may define distal end 502A of stent 502. One end of second row 504A may define proximal end 502A of stent 502. The ends of first row 504A and second row 504B defining distal end 502A and proximal end 502A, respectively, may each define a reference plane orthogonal to longitudinal axis 104, e.g., such that stent 502 defines a cylindrical perimeter. For example, first row 504A and second 504B may form flat ends of stent 502 at distal end 502A and proximal end 502B. Flat ends at distal end 502A and proximal end 502B may facilitate deployment of stent 502 within a body lumen of the patient (e.g., blood vessel 106 of FIG. 1).

First row 504A and second row 504B may include struts 110 of varying lengths, e.g., to facilitate transition between first row 504A, second row 504B and rows 506 of struts 110. Frist row 504A and/or second row 504B may include a different number of struts 110 than rows 506. For example, as illustrated in FIG. 7, first row 504A may include struts 110 of increasing lengths such that an end of first row 504A configured to be connected to row 506 defines a pitch the same as or similar to the pitch of row 506. In such examples, connectors 112 connecting first row 504A to row 506, connecting longitudinally adjacent rows 506, and connecting row 506 to second row 504B may be of the same or similar longitudinal lengths, which may cause stent 502 to maintain a same strength and/or flexibility as another stent without rows of struts orthogonal to the longitudinal axis of the stent.

Each of rows 506 may extend around longitudinal axis 104. Each of rows 506 may define at least a portion of the helical pattern of the stent 502. For example, each of rows 506 may define a helical axis 503 offset from longitudinal axis 104. Helical axis 503 may be offset from longitudinal axis 104 by a helical angle 505. Rows 506 may define uniform or varying helical angles 505 along the longitudinal length of stent 502. For example, helical angles 505 of rows 506 may increase from distal end 502A to proximal end 502B, or vice versa. Each of rows 506 may correspond to one complete revolution of the helical or spiral pattern around the perimeter of stent 502. Longitudinally adjacent rows 506 of struts 110 may be connected via connectors 112, e.g., as previously described herein with respect to example stent 102 of any of FIGS. 1-5. For example, at least some of the peaks of one row 506 of struts 102 may be coupled to at least some of the valleys of a longitudinally adjacent row 506 of struts 102 via connectors 112.

The placement of connectors 112 around the perimeter of stent 502 may be independent from the helical or spiral pattern of rows 506. Connectors 112 may connect one out of every plurality of apices within one row 506 to apices within a longitudinally adjacent row 506. Connectors 112 may be connected to one out of every two, three, four, or five or more apices. A number of connectors 112 within stent 502 may be a balance between stiffness, integrity, flexibility, and apposition capabilities of stent 502.

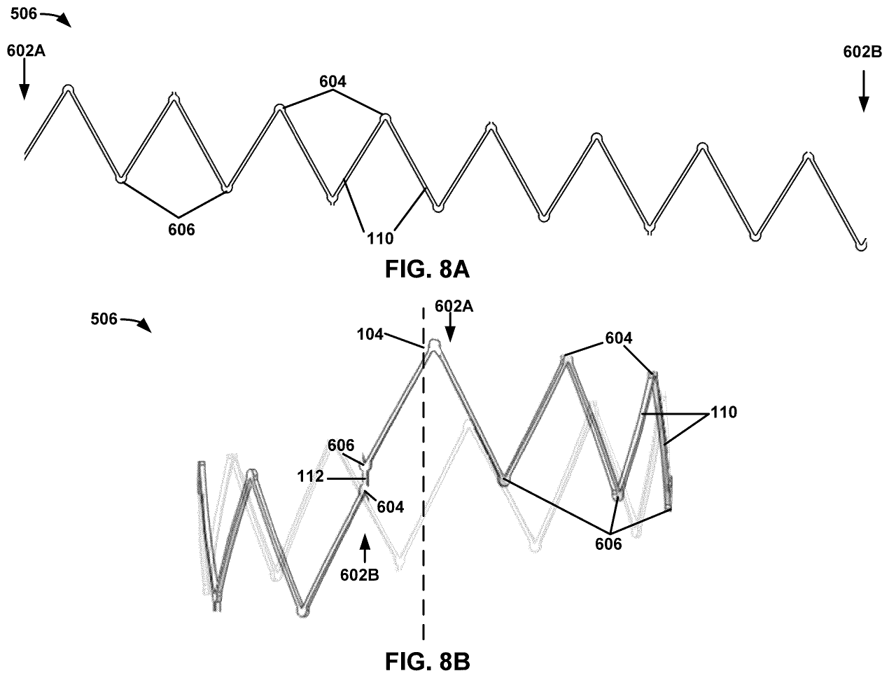

FIG. 8A is a conceptual diagram illustrating an example row 506 of struts 110 of FIG. 6, the example row 506 has been longitudinally cut and laid flat. FIG. 8B is a conceptual diagram illustrating another view of the example row 506 of struts 110 of FIG. 8A. Row 506 may extend from a first end 602A to a second end 602B. Struts 110 within row 506 may define a plurality of apices, the plurality of apices including a plurality of peaks 604 and a plurality of valleys 606.

In some examples, each row 506 may define a helical or spiral pattern independent of other rows 506 within stent 502. For example, as illustrated in FIG. 8B, first end 602A of row 506 may be connected to second end 602B (e.g., a valley 606 at first end 602A may be connected to a peak 604 at second end 602B) via connector 112 to form a complete helical or spiral pattern. The complete helical or spiral pattern defined by row 506 may make one complete revolution around the perimeter of stent 502. Multiple rows 506 may be coupled via connectors 112 to form the body of stent 502 extending between first row 504A and second row 504B.

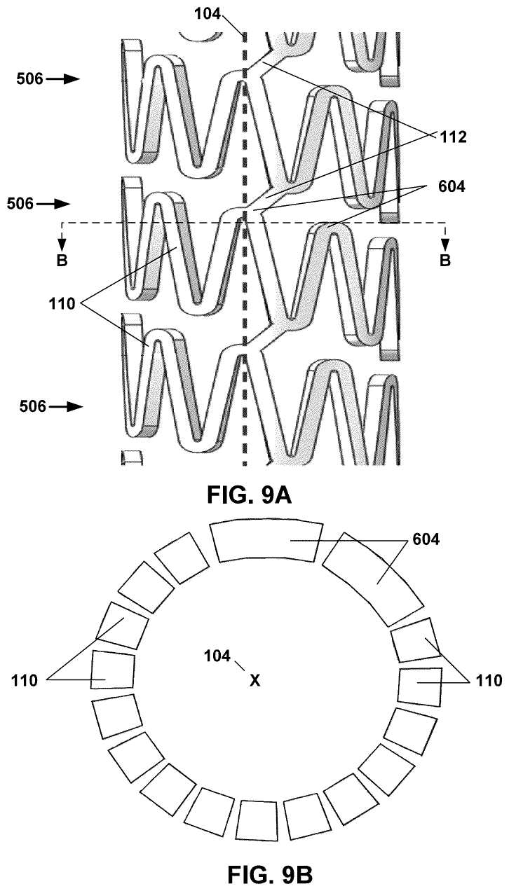

FIG. 9A is a conceptual diagram illustrating an example unit cell of FIG. 6 in the collapsed configuration. FIG. 9B is a cross-sectional diagram illustrating a cross-section view of the example unit cell of FIG. 9B, the cross-section being taken along line B-B of FIG. 9A.

Apices within each row 506 may be disposed along the helical path defined by row 506. For example, as illustrated in FIG. 9A, peaks 604 within row 506 may be longitudinally offset by at least the helical pitch of row 506. Similarly, valleys 606 within row 506 may be longitudinally offset by at least the helical pitch of row 506. One or more peaks of one row 506 may be connected to one or more peaks or valleys of an adjacent row 506 (e.g., a distal row 506). In some examples, where the one or more peaks of one row 506 are connected to one or more valleys of an adjacent row 506, one end of the row 506 may define a partial apex (e.g., half of an apex), e.g., to facilitate a continuous connection between adjacent rows 506. Stents 502 with peak-to-valley connections may be capable of expanding to a greater diameter while maintaining radial support and flexibility than an otherwise identical stent with peak-to-peak connections.

As illustrated in FIG. 9A, peaks 604 of each row 506 of struts 110 may be circumferentially offset from valleys of a longitudinally adjacent row 506 of struts 110. The circumferential offset may inhibit interference between struts 110 of longitudinally adjacent rows 506 when stent 502 is compressed. Connectors 112 may extend along reference axes offset from longitudinal axis 104 to connect longitudinally adjacent rows 506 and/or ends 602A, 602B of rows 506.

When stent 502 is in collapsed configuration, peaks 604 within row 506 may be longitudinally offset and valleys 606 within row 506 may be longitudinally offset. Longitudinal offset of peaks 604 and of valleys 606 may reduce an outer diameter of row 506 in collapsed configuration, e.g., by staggering peaks 604 and valleys 606 at different longitudinal positions and prevent overlapping of peaks 604 and/or valleys 606 when stent 502 is collapsed. For example, as illustrated in FIG. 9B, peaks 604 and valleys 606 are longitudinally staggered such that all peaks 604 and/or all valleys 606 defined by a row 506 of struts 110 are not disposed at a same longitudinal position. The reduced outer diameter may allow stent 502 to define a smaller outer diameter than an identical stent without longitudinally offset apices or may include struts 110 with greater widths and/or diameters than another stent without longitudinally offset apices.

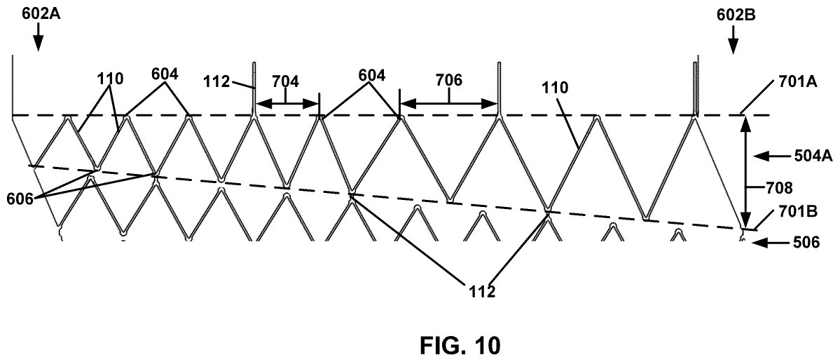

FIG. 10 is a conceptual diagram illustrating an example end row of struts 110 of stent 502 of FIG. 6, the example row has been longitudinally cut and laid flat. While FIG. 10 illustrates first row 504A, second row 504B may exhibit same or similar dimensions and geometry. First row 504A extends from first end 602A to second end 602B. Struts 110 may extend between a first end 701A of first row 504A (e.g., distal end 502A of stent 502) and a second end 701B of first row 504A. Struts 110 may define a plurality of peaks 604 at first end 701A and a plurality of valleys 606 at second end 701B. Struts 110 within first row 504A may define a different number of peaks 604 and valleys 606. For example, struts 110 within first row 504A may define more valleys 606 than peaks 604. Apices of first row 504A may alternate between peaks 604 and valleys 606 from first end 602A to second end 602B. In some examples, as illustrated in FIG. 10, struts 110 of first row 504A defines a half apex (e.g., at or around second end 602B). The half apex may facilitate transition of struts 110 from first row 504A to the longitudinally adjacent row 506.

Struts 110 may define varying longitudinal lengths from first end 602A to second end 602B. Second end 701B may define a helical path around the perimeter of stent 502. The helical path may define a same or similar helical angle as row 506, e.g., to facilitate transition between first row 504A and the longitudinally adjacent row 506. One or more valleys 606 of first row 504A may be connected to one or more peaks of the longitudinally adjacent row 506 via one or more connectors 112. Struts 110 within first row 504A may define uniform or varying apex angles between first end 602A and second end 602B. For example, as illustrated in FIG. 10, struts 110 may define peaks 604 and valleys 606 with a first apex angle at or around first end 602A and peaks 604 and valleys 606 with a second apex angle at or around second end 602B.

In some examples, apex angles may be uniform within first row 504A from first end 602A to second end 602B, e.g., to maintain cause stent 502 to maintain consistent strength and/or flexibility around the entire perimeter of stent 502. In such examples, struts 110 may define gaps between peaks 604 with different widths 704, 706 facilitate the placement and number of apices defined by struts 110 without adjusting the apex angles of the apices. Widths 704, 706 may be measured along a reference axis orthogonal to longitudinal axis 104. Width 706 may depend on a total width of first row 504A from first end 602A to second end 602B and/or a number of apices and/or half-apices defined by struts 110 within first row 504A. A combination of all widths 704, 706 from first end 602A to second end 602B may be equivalent to an integer number of widths 704, e.g., to compensate for the presence of any half apices within first row 504A. For example, as illustrated in FIG. 10, struts 110 of first row 504A define four gaps between peaks 604 with width 706 and four gaps between peaks 604 with width 704. Gaps between peaks 604 with width 706 may be disposed at or around one end (e.g., second end 602B) of first row 504A or may be distributed between first end 602A and second end 602B. For example, the widths of gaps between peaks 604 may alternate between width 704 and width 706.

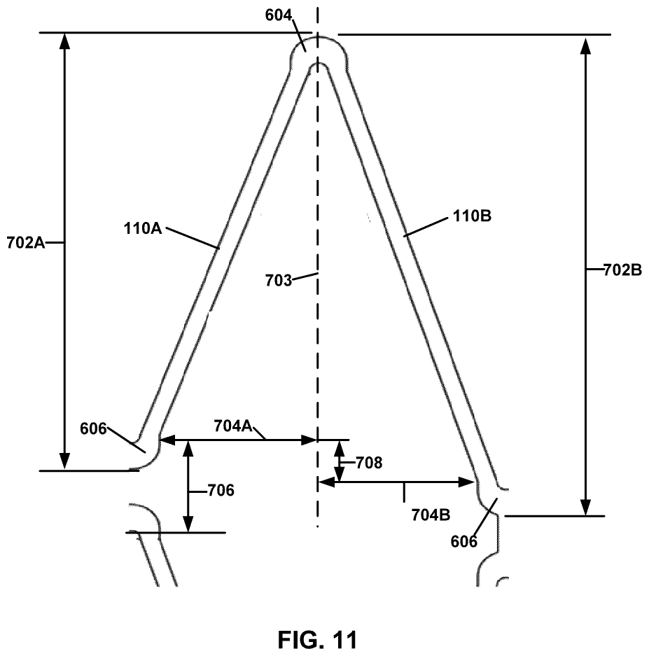

FIG. 11 is a conceptual diagram illustrating an example pair of struts 110 of the example row 506 of struts of FIG. 8A. Struts 110 may include a first strut 110A and a second strut 110B. Each of struts 110A, 110B may be connected to a peak 604 and a valley 606. Struts 110A, 110B may be commonly connected at peak 604 disposed between struts 110A, 110B. In other examples, circumferentially adjacent struts 110 may be commonly connected at valley 606 disposed between struts 110. Struts 110A, 110B, may be symmetrical about a longitudinal axis 703 extending through peak 604. Longitudinal axis 703 may be parallel to or may be offset from longitudinal axis 104 of stent 502, as illustrated in FIG. 6.

Strut 110A may define a height 702A along longitudinal axis 104 and define a maximum width 704A between strut 110A and longitudinal axis 703 along a reference axis orthogonal to longitudinal axis 703. The maximum width 704A may be measured at an end of strut 110A connected to valley 606. Strut 110B may define a height 702B along longitudinal axis 104 and define a maximum width 704B between strut 110B and longitudinal axis 703 along a reference axis orthogonal to longitudinal axis 703. Width 704B may be measured at an end of strut 110B connected to valley 606.

As illustrated in FIG. 11, height 702A of strut 110A may be different from height 702B of strut 110B, e.g., to maintain the helical or spiral pattern defined by row 506 of struts 110. Height 702A may be greater than or less than height 702B depending on the direction of winding of the helical or spiral pattern defined by row 506. For example, as illustrated in FIGS. 6-11, height 702A may be less than height 702B to support a counterclockwise rotation of the helix or spiral defined by row 506 about longitudinal axis 104. Height 702A and height 702B may differ by distance 708. Each valley 606 defined by one of struts 110A, 110B may be separated from peak 604 of a longitudinally adjacent row 506 by distance 706. Similarly, peak 605 defined by struts 110A, 110B may be longitudinally offset from valley 606 of a longitudinally adjacent row 506 by distance 706.

Width 704A may be of a same or different value as width 704B. When width 704A is equal to width 704B, each of struts 110A and 110B may be offset from longitudinal axis 703 by a same angle. The offset angle may be determined based on a circumference of row 506 and a number of peaks 604 within row 506. For example, struts 110 in row 506 with more peaks 604 may define smaller offset angles than struts 110 in row 506 with fewer peaks 604. The apex angle of peak 604 may be a sum of the offset angles for struts 110A, 110B defining peak 604.

The helical pattern of row 506 may be defined by a number of peaks 604 per row, a pitch of the helical winding of the helical pattern, stent 502 diameter, and longitudinal offset between apices of adjacent rows 506 (e.g., distance 706).

Height 706A may be calculated via equation (1) and height 706B may be calculated via equation (2). The width between circumferentially adjacent valleys 606 along row 506 may be calculated via equation (3). The variables for equations (1)-(3) are described in Table 1 below.

$${L}_{1} = \sqrt{{( P - Δ L )}^{2} + {( \frac{W}{2} )}^{2}} \qquad ( 1 )$$

$${L}_{2} = \sqrt{{( P - Δ L + \frac{P}{N} )}^{2} + {( \frac{W}{2} )}^{2}} \qquad ( 2 )$$

$$W = \frac{π D}{N} \qquad ( 3 )$$

TABLE 1

| Sign | Meaning |
|---|---|
| L₁ | Height 706A of strut 110A |
| L₂ | Height 706B of strut 110B |
| P | Pitch of the helical winding of the helical pattern |
| ΔL | Longitudinally offset distance 706 between adjacent rows 506 |
| W | Width between circumferentially adjacent valleys 606 |
| N | Number of peaks 604 per row 506 |
| D | Outer diameter of stent 502 |

Based on the desired parameter values for the helical pattern of rows 506 within stent 502 (e.g., number of peaks 604, diameter of stent 502, pitch of the helical winding, longitudinal offset between apices), a manufacturing system may determine the dimensions and placement for struts 110, connectors 112, peaks 604, and valleys 606 about an elongated body and may remove material from the elongated body, e.g., via a cutting instrument, via a laser-cutting technique, to form stent 502 with the desired parameters.

While stent 102 and stent 502 have primarily been described separately above, other example stents may include one or more elements from both stent 102 and 502. In some examples, an example stent includes rows 114 defining a helical pattern, e.g., as described herein within respect to rows 506 of stent 502. In some examples, an example of stent 502 may include rows 506 defining unit cells (e.g., unit cell 200) and apices defining different apex angles within each unit cell. In some examples, a stent includes two or more sections along longitudinal axis 104, wherein each section includes rows of struts 110 having the same dimensions and/or features as one of stents 102, 502 as described herein. For example, a first section of the stent may include rows 114 of struts 110 and a second section of the stent may include rows 504A, 504B, and/or rows 506.

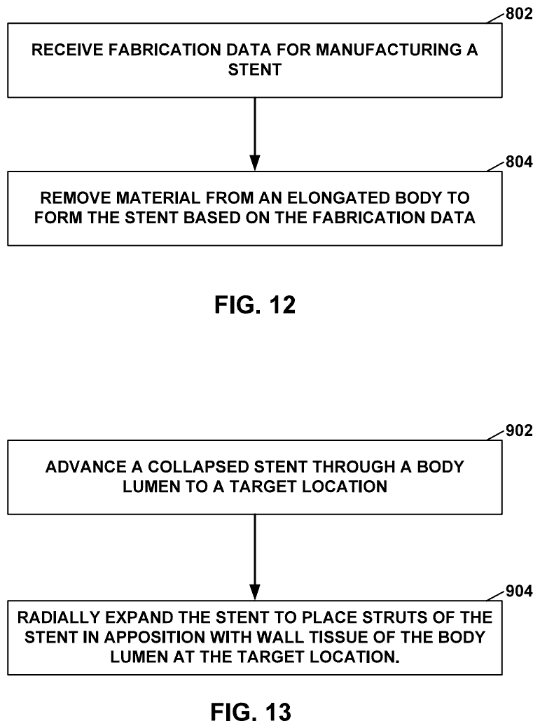

FIG. 12 is a flow chart illustrating an example process of manufacturing an example stent. While the example process illustrated in FIG. 12 is described primarily as being performed by a subtractive manufacturing assembly, the example process may be performed be a manufacturer and/or by other manufacturing systems (e.g., an additive manufacturing assembly) to manufacture the example stents described in this disclosure. For example, an additive manufacturing assembly may determine the placement of elements of stents about a reference elongated body, e.g., in accordance with the example processes discussed below, and may form the elements of the stent via an additive manufacturing technique.

A subtractive manufacturing assembly may receive fabrication data for manufacturing a stent (e.g., stent 102, stent 502) (802). The fabrication data may indicate the placement of struts 110 and connectors 112 defining the stent around a circumference of an elongated body used to form the stent. In some examples, the subtractive manufacturing assembly receives the fabrication data, e.g., from a computer device, via user input from a manufacturer. In some examples, the subtractive manufacturing assembly may determine the placement of struts 110 and connectors 112 and generate the fabrication data.

Fabrication data may include placement of struts 110 and connectors 112 along a longitudinal length of the elongated body and around the outer perimeter of the elongated body. The longitudinal axis of the elongated body may be the same as the longitudinal axis of the stent (e.g., longitudinal axis 104). Portions of the elongated body outside of struts 110 and connectors 112 to be removed by the subtractive manufacturing assembly. Struts 110 and connectors 112 may define a single, continuous stent. The fabrication data may indicate a cross-section for struts 110 and connectors 112 (e.g., circular, oval, rectangular, trapezoidal, pentagonal, hexagonal, or other polygonal shapes).

Struts 110 may define a plurality of rows (e.g., rows 114, 504A, 504B, 506), each row extending around the circumference of the elongated body. When the subtractive manufacturing assembly forms the stent from the elongated body, the struts of the plurality of rows may form an outer perimeter of the stent. Within each row, struts 110 may define a plurality of apices, each apex connecting two circumferentially adjacent struts. Apices may include peaks (e.g., peaks 204A, 304, 312, 604) pointing towards a distal end of the stent and valleys (e.g., valleys 204B, 306, 314, 606) pointing towards a proximal end of the stent. The fabrication data may include struts 110 defining apices with alternating peaks and valleys around the perimeter of the elongated body.

Each row of struts 110 may be connected to one or more longitudinally adjacent rows of struts 110 via connectors 112. The Fabrication data may define longitudinal offsets between longitudinally adjacent rows of struts 110 and the lengths (e.g., longitudinal lengths) of connectors 112. Connectors 112 may extend parallel to the longitudinal axis or may be offset from the longitudinal axis.

As defined by the placements of struts 110 and connectors 112, apices of longitudinally adjacent rows of struts 110 may be circumferentially offset. The circumferential offset may allow longitudinally adjacent apices to longitudinally overlap without circumferentially overlapping, e.g., in response to compression of the stent. The circumferential offset may consequently reduce or inhibit unintended interference between struts 110 and/or apices of longitudinally adjacent rows of struts 110 in response to compression of the stent, which may increase apposition of struts 110 to a surface of a body lumen of the patient and/or increase efficacy of the stent.

In some examples, as illustrated in FIGS. 1-5, unit cells 200 defined by struts 110 and connectors 120 may include apices with different apex angles within each unit cell 200. The fabrication data may include the placement of all struts 110 and connectors 120 along the entire stent. In some examples, the fabrication data includes the placement of struts 110 and connectors 120 within a single unit cell 200. The subtractive manufacturing assembly may then replicate the single unit cell 200 to define the entire stent, e.g., in accordance with instructions stored in the subtractive manufacturing assembly.

In some examples, as illustrated in FIGS. 6-11, rows (e.g., rows 506) of struts 110 may define a helical or spiral pattern. The fabrication data may include instructions to form a stent within the helical or spiral pattern defined by rows 506. In some examples, the fabrication data may include placements for all struts 110 and connectors 120 within the stent and defining the helical or spiral pattern. In some examples, the fabrication data includes a single row 506 of struts 110 and the subtractive manufacturing assembly replicates the singled row 506 at different locations along the elongated body to generate the placement of struts 110 and connectors 112 on the stent. In some examples, the fabrication data includes perimeter values for the helical pattern including, but are not limited to, the number of peaks 604 per row, the pitch of the helical winding of the helical pattern, and the longitudinal offset between apices of adjacent rows. The subtractive assembly may, based on the fabrication data in conjunction with an outer diameter of the elongated body, determine the dimensions and placements of struts 110 and connectors 112, e.g., based at least in part on equations 1-3.

The subtractive manufacturing assembly may remove material from the elongated body form the stent based on the fabrication data (804). The subtractive manufacturing assembly may remove material to form struts 110 and connectors 112. The subtractive manufacturing assembly may determine the placements of struts 110 and connectors 112 based on the fabrication data and may remove material from the elongated body to form struts 110 and connectors 112. The subtractive manufacturing assembly may remove the material via one or more techniques including, but are not limited to, a laser-cutting technique, a cutting instrument (e.g., a bladed instrument), or application of one or more chemicals.

FIG. 13 is a flow chart illustrating an example process of deploying an example stent within a patient. While the example process illustrated in FIG. 13 is primarily described with respect to deployment of the stent within a blood vessel (e.g., blood vessel 106) of the patient, the example process may be applied to deploy an example stent within another body lumen of the patient, e.g., portions of the gastrointestinal tract, ureters of the patient, or the like.

A clinician may advance a collapsed stent through a body lumen (e.g., blood vessel 106) to a target location (902). The clinician may couple the stent in the collapsed configuration to a delivery system (e.g., to a delivery catheter) and advance the delivery system within the patient (e.g., within vasculature of the patient) to the target location. The clinician may visualize the position of the stent within the patient via one or more imaging techniques including, but are not limited to, fluoroscopy.

The delivery system may retain the stent in the collapsed configuration. In the collapsed configuration, the stent may define a reduced outer diameter than the stent in an expanded configuration. The collapsed configuration may facilitate navigation of the stent within the patient, e.g., within the vasculature of the patient. The stent may be flexible in the collapsed configuration to facilitate navigation of the stent around curvatures within the vasculature of the patient to the target location.

The clinician may radially expand the stent to place struts 110 of the stent in apposition with wall tissue of the body lumen at the target location. (904). For example, the clinician may radially expand the stent to the expanded configuration to place struts 110 in apposition with vessel wall 108 of blood vessel 106. When the expanded stent is placed in apposition with the body lumen wall, the stent may retain or expand the diameter of the body lumen wall, thereby facilitate flow of fluids through the body lumen.

In some examples, where the stent is self-expanding, the clinician may release the stent from the delivery system at the target location and the stent may automatically expand from the collapsed configuration to the expanded configuration. In some examples, the clinician may expand the stent via expansion of an expandable member (e.g., an occlusion element, a balloon, a basket, or the like) of the delivery element to cause the stent to expand to the expanded configuration.

The target location may be disposed at or around a curvature in the body lumen and at least a portion of the expanded stent may be disposed within the curvature. In some examples, movement by the patient may cause body lumen to at least temporarily bend. The stent may bend and conform to the curvature, e.g., to maintain apposition with the body lumen wall. At least a portion of stent may compress and another portion of the stent may elongate, to cause the stent to bend. When the stent is at least partially compressed, apices of longitudinally adjacent rows of struts 110 may longitudinally overlap. The apices of longitudinally adjacent rows may be circumferentially offset (e.g., as discussed in greater detail above) to inhibit interference between struts and apices of longitudinally adjacent rows during compression of the stent. The circumferential offset between apices of adjacent rows may thereby increase apposition of struts 110 to the body lumen wall and/or increase efficacy of the stent compared to an otherwise identical stent with circumferentially aligned apices.

This disclosure described the following examples:

Example 1: a stent comprising: a plurality of rows of struts extending along the longitudinal axis, each row of struts of the plurality of rows of struts extending around the longitudinal axis; and a plurality of rows of connectors, each row of connectors of the plurality of connectors extending between longitudinally adjacent rows of struts; wherein the plurality of rows of struts and the plurality of rows of connectors define a plurality of unit cells, wherein each unit cell is defined by: a first set of struts of a first row of struts of the plurality of rows of struts, a second set of struts of a second row of struts of the plurality of rows of struts, and two circumferentially adjacent connectors of a row of connectors of the plurality of rows, the row of connectors being between the first row of struts and the second row of struts along the longitudinal axis, wherein within each unit cell, the first set of struts define a first plurality of apices and the second set of struts define a second plurality of apices, wherein within each unit cell, a first strut and a second strut of the first set of struts define a first apex with a first apex angle, and the second strut and a third strut of the first set of struts define a second apex with a second apex angle, the second apex angle being less than the first apex angle, and wherein within each unit cell, a fourth strut and a fifth strut of the second set of struts define a third apex with the first apex angle, and the fifth strut and the sixth strut define a fourth apex with the second apex angle.

Example 2: the stent of example 1, wherein the stent is configured to be compressed along the longitudinal axis, wherein a longitudinally distance between longitudinally adjacent rows of struts is reduced when the stent is in a compressed configuration, and wherein the second apex angle is less than the first apex angle to reduce contact between the first plurality of apices and the second plurality of apices when the stent is in the compressed configuration.

Example 3: the stent of any of examples 1 and 2, wherein each unit cell extends around the longitudinal axis from a first end to a second end, wherein a first connector of the two circumferentially adjacent connectors defines the first end, wherein a second connector of the two circumferentially adjacent connectors define the second end, wherein the first strut is connected to the first connector, and wherein the fourth strut is connected to the second connector.

Example 4: the stent of any of examples 1-3, wherein each connector of the plurality of rows of connectors extends parallel to the longitudinal axis.

Example 5: the stent of any of examples 1-4, wherein for each unit cell, the first plurality of apices comprises a first plurality of peaks extending towards a distal end of the stent and a first plurality of valleys extending towards a proximal end of the stent, wherein for each unit cell, the second plurality of apices comprises a second plurality of peaks extending towards the distal end of the stent and a second plurality of valleys extending towards the proximal end of the stent, and wherein for each unit cell, the second apex angle is less than the first apex angle to cause each peak of the first plurality of peaks to be circumferentially offset from each valley of the second plurality of valleys and to cause each valley of the first plurality of valleys to be circumferentially offset from each peak of the second plurality of peaks.

Example 6: the stent of example 5, wherein each peak of the first plurality of peaks is defined by a peak angle, wherein each valley of the first plurality of valleys is defined by a valley angle, wherein the peak angle of at least one peak of the first plurality of peaks is equal to the first apex angle, wherein the peak angles of a remaining number of peaks of the first plurality of peaks is equal to the second apex angle, wherein the valley angle of at least one valley of the first plurality of valleys is equal to the first apex angle, and wherein the valley angles of a remaining number of valleys of the first plurality of valleys is equal to the second apex angle.

Example 7: the stent of any of examples 1-6, wherein the first apex angle is less than or equal to 90 degrees.

Example 8: the stent of any of examples 1-7, wherein a difference between the first apex angle and the second apex angle is less than or equal to 20 degrees.

Example 9: the stent of any of examples 1-8, wherein each row of struts of the plurality of rows of struts is centrosymmetric about the longitudinal axis.

Example 10: the stent of example 9, wherein each row of connectors of the plurality of rows of connectors is centrosymmetric about the longitudinal axis.

Example 11: the stent of any of examples 1-10, wherein the stent is formed via laser-cutting.

Example 12: the stent of any of examples 1-11, wherein the plurality of rows of struts defines a helical pattern along the longitudinal axis from a distal end of the stent to a proximal end of the stent.

Example 13: the stent of any of examples 1-12, wherein one or more rows of the plurality of rows of struts each define a helical pattern extend along the longitudinal axis and around the longitudinal axis.

Example 14: the stent of any of examples 12 and 13, wherein within one or more rows of the plurality of rows of struts, struts within the one or more rows define varying longitudinal lengths along longitudinal axis.

Example 15: the stent of any of examples 1-14, wherein each apex of the first plurality of apices is longitudinally offset and circumferentially offset from every apex of the second plurality of apices.

Example 16: a method comprising: receiving, by a subtractive manufacturing assembly, fabrication data for manufacturing a stent, the stent comprising: a plurality of rows of struts extending along the longitudinal axis, each row of struts of the plurality of rows of struts extending around the longitudinal axis; and a plurality of rows of connectors, each row of connectors of the plurality of connectors extending between longitudinally adjacent rows of struts; wherein the plurality of rows of struts and the plurality of rows of connectors define a plurality of unit cells, wherein each unit cell is defined by: a first set of struts of a first row of struts of the plurality of rows of struts, a second set of struts of a second row of struts of the plurality of rows of struts, and two circumferentially adjacent connectors of a row of connectors of the plurality of rows, the row of connectors being between the first row of struts and the second row of struts along the longitudinal axis, wherein within each unit cell, the first set of struts define a first plurality of apices and the second set of struts define a second plurality of apices, wherein within each unit cell, a first strut and a second strut of the first set of struts define a first apex with a first apex angle, and the second strut and a third strut of the first set of struts define a second apex with a second apex angle, the second apex angle being less than the first apex angle, and wherein within each unit cell, a fourth strut and a fifth strut of the second set of struts define a third apex with the first apex angle, and the fifth strut and the sixth strut define a fourth apex with the second apex angle; and removing, by the subtractive manufacturing assembly, material from an elongated body to form the stent.

Example 17: the method of example 16, wherein the stent is configured to be compressed along the longitudinal axis, wherein a longitudinally distance between longitudinally adjacent rows of struts is reduced when the stent is in a compressed configuration, and wherein the second apex angle is less than the first apex angle to reduce contact between the first plurality of apices and the second plurality of apices when the stent is in the compressed configuration.

Example 18: the method of example 17, wherein each unit cell extends around the longitudinal axis from a first end to a second end, wherein a first connector of the two circumferentially adjacent connectors define the first end, wherein a second connector of the two circumferentially adjacent connectors define the second end, wherein the first strut is connected to the first connector, and wherein the fourth strut is connected to the second connector.

Example 19: the method of any of examples 16-18, wherein each connector of the plurality of rows of connectors extends along the longitudinal axis.

Example 20: the method of any of examples 16-19, wherein for each unit cell, the first plurality of apices comprises a first plurality of peaks extending towards a distal end of the stent and a first plurality of valleys extending towards a proximal end of the stent, wherein for each unit cell, the second plurality of apices comprises a second plurality of peaks extending towards the distal end of the stent and a second plurality of valleys extending towards the proximal end of the stent, and wherein for each unit cell, the second apex angle is less than the first apex angle to cause each peak of the first plurality of peaks to be circumferentially offset from each valley of the second plurality of valleys and to cause each valley of the first plurality of valleys to be circumferentially offset from each peak of the second plurality of peaks.

Example 21: the method of example 20, wherein each peak of the first plurality of peaks is defined by a peak angle, wherein each valley of the first plurality of valleys is defined by a valley angle, wherein the peak angle of at least one peak of the first plurality of peaks is equal to the first apex angle, wherein the peak angles of a remaining number of peaks of the first plurality of peaks is equal to the second apex angle, wherein the valley angle of at least one valley of the first plurality of valleys is equal to the first apex angle, and wherein the valley angles of a remaining number of valleys of the first plurality of valleys is equal to the second apex angle.

Example 22: the method of any of examples 16-21, wherein the first apex angle is less than or equal to 90 degrees.

Example 23: the method of any of examples 16-22, wherein a difference between the first apex angle and the second apex angle is less than or equal to 20 degrees.

Example 24: the method of any of examples 16-23, wherein each row of struts of the plurality of rows of struts is centrosymmetric about the longitudinal axis.

Example 25: the method of claim any of examples 16-24, wherein each row of connectors of the plurality of rows of connectors is centrosymmetric about the longitudinal axis.

Example 26: a method comprising: advancing a stent within a body lumen of a patient to a target location, wherein the stent comprises: a plurality of rows of struts extending along the longitudinal axis, each row of struts of the plurality of rows of struts extending around the longitudinal axis; and a plurality of rows of connectors, each row of connectors of the plurality of connectors extending between longitudinally adjacent rows of struts; wherein the plurality of rows of struts and the plurality of rows of connectors define a plurality of unit cells, wherein each unit cell is defined by: a first set of struts of a first row of struts of the plurality of rows of struts, a second set of struts of a second row of struts of the plurality of rows of struts, and two circumferentially adjacent connectors of a row of connectors of the plurality of rows, the row of connectors being between the first row of struts and the second row of struts along the longitudinal axis, wherein within each unit cell, the first set of struts define a first plurality of apices and the second set of struts define a second plurality of apices, wherein within each unit cell, a first strut and a second strut of the first set of struts define a first apex with a first apex angle, and the second strut and a third strut of the first set of struts define a second apex with a second apex angle, the second apex angle being less than the first apex angle, and wherein within each unit cell, a fourth strut and a fifth strut of the second set of struts define a third apex with the first apex angle, and the fifth strut and the sixth strut define a fourth apex with the second apex angle; and expanding the stent radially outwards of the longitudinal axis at the target location to place the struts of the plurality of rows of struts in apposition with a surface of the body lumen at the target location.

Example 27: the method of example 26, wherein the stent is configured to be compressed along the longitudinal axis, wherein a longitudinally distance between longitudinally adjacent rows of struts is reduced when the stent is in a compressed configuration, and wherein the second apex angle is less than the first apex angle to reduce contact between the first plurality of apices and the second plurality of apices when the stent is in the compressed configuration.

Example 28: the method of example 27, wherein when the stent is in the expanded configuration, the stent is configured to bend about a reference plane orthogonal to the longitudinal axis in response to curvature of the body lumen at the target location, wherein bending of the stent about the reference plane causes at least a portion of the stent to transition into the compressed configuration, and wherein the second apex angle is less than the first apex angle to reduce contact between the first plurality of apices and the second plurality of apices when the stent bends about the reference plane.

Example 29: the method of any of examples 27 and 28, wherein each peak of the first plurality of peaks is defined by a peak angle, wherein each valley of the first plurality of valleys is defined by a valley angle, wherein the peak angle of at least one peak of the first plurality of peaks is equal to the first apex angle, wherein the peak angles of a remaining number of peaks of the first plurality of peaks is equal to the second apex angle, wherein the valley angle of at least one valley of the first plurality of valleys is equal to the first apex angle, and wherein the valley angles of a remaining number of valleys of the first plurality of valleys is equal to the second apex angle.

Example 30: the method of any of examples 26-29, wherein each connector of the plurality of rows of connectors extends parallel to the longitudinal axis.

Example 31: the method of any of examples 26-30, wherein the first apex angle is less than or equal to 90 degrees.

Example 32: the method of any of examples 26-31, wherein a different between the first apex angle and the second apex angle is less than or equal to 20 degrees.

Example 33: the method of any of examples 26-32, wherein each row of struts of the plurality of rows of struts is centrosymmetric about the longitudinal axis.

Various aspects of the disclosure have been described. These and other aspects are within the scope of the following claims.

What is claimed is:

1. A stent comprising:
a plurality of rows of struts extending along the longitudinal axis, each row of struts of the plurality of rows of struts extending around the longitudinal axis; and
a plurality of rows of connectors, each row of connectors of the plurality of connectors extending between longitudinally adjacent rows of struts,
wherein the plurality of rows of struts and the plurality of rows of connectors define a plurality of unit cells,
wherein each unit cell is defined by:
a first set of struts of a first row of struts of the plurality of rows of struts,
a second set of struts of a second row of struts of the plurality of rows of struts, and
two circumferentially adjacent connectors of a row of connectors of the plurality of rows, the row of connectors being between the first row of struts and the second row of struts along the longitudinal axis,
wherein within each unit cell, the first set of struts define a first plurality of apices and the second set of struts define a second plurality of apices,
wherein within each unit cell, a first strut and a second strut of the first set of struts define a first apex with a first apex angle, and the second strut and a third strut of the first set of struts define a second apex with a second apex angle, the second apex angle being less than the first apex angle, and
wherein within each unit cell, a fourth strut and a fifth strut of the second set of struts define a third apex with the first apex angle, and the fifth strut and the sixth strut define a fourth apex with the second apex angle.

2. The stent of claim 1, wherein the stent is configured to be compressed along the longitudinal axis, wherein a longitudinally distance between longitudinally adjacent rows of struts is reduced when the stent is in a compressed configuration, and wherein the second apex angle is less than the first apex angle to reduce contact between the first plurality of apices and the second plurality of apices when the stent is in the compressed configuration.

3. The stent of any of claim 1,
wherein each unit cell extends around the longitudinal axis from a first end to a second end,
wherein a first connector of the two circumferentially adjacent connectors defines the first end,
wherein a second connector of the two circumferentially adjacent connectors define the second end,
wherein the first strut is connected to the first connector, and
wherein the fourth strut is connected to the second connector.

4. The stent of claim 1, wherein each connector of the plurality of rows of connectors extends parallel to the longitudinal axis.

5. The stent of claim 1,
wherein for each unit cell, the first plurality of apices comprises a first plurality of peaks extending towards a distal end of the stent and a first plurality of valleys extending towards a proximal end of the stent,
wherein for each unit cell, the second plurality of apices comprises a second plurality of peaks extending towards the distal end of the stent and a second plurality of valleys extending towards the proximal end of the stent, and
wherein for each unit cell, the second apex angle is less than the first apex angle to cause each peak of the first plurality of peaks to be circumferentially offset from each valley of the second plurality of valleys and to cause each valley of the first plurality of valleys to be circumferentially offset from each peak of the second plurality of peaks.

6. The stent of claim 5,
wherein each peak of the first plurality of peaks is defined by a peak angle, wherein each valley of the first plurality of valleys is defined by a valley angle,
wherein the peak angle of at least one peak of the first plurality of peaks is equal to the first apex angle,
wherein the peak angles of a remaining number of peaks of the first plurality of peaks is equal to the second apex angle,
wherein the valley angle of at least one valley of the first plurality of valleys is equal to the first apex angle, and
wherein the valley angles of a remaining number of valleys of the first plurality of valleys is equal to the second apex angle.

7. The stent of claim 1, wherein the first apex angle is less than or equal to 90 degrees.

8. The stent of claim 1, wherein a difference between the first apex angle and the second apex angle is less than or equal to 20 degrees.

9. The stent of claim 1, wherein each row of struts of the plurality of rows of struts is centrosymmetric about the longitudinal axis.

10. The stent of claim 9, wherein each row of connectors of the plurality of rows of connectors is centrosymmetric about the longitudinal axis.

11. The stent of any of claim 1, wherein the stent is formed via laser-cutting.

12. The stent of claim 1, wherein the plurality of rows of struts defines a helical pattern along the longitudinal axis from a distal end of the stent to a proximal end of the stent.

13. The stent of claim 1, wherein one or more rows of the plurality of rows of struts each define a helical pattern extend along the longitudinal axis and around the longitudinal axis.

14. The stent of claim 13, wherein within one or more rows of the plurality of rows of struts, struts within the one or more rows define varying longitudinal lengths along longitudinal axis.

15. The stent of claim 1, wherein each apex of the first plurality of apices is longitudinally offset and circumferentially offset from every apex of the second plurality of apices.
